(19) United States
(12) Patent Application Publication
(10) Pub. No.: US 2023/0238133 A1
(43) Pub. Date: Jul. 27, 2023

(54) ECG SEARCH AND INTERPRETATION BASED ON A DUAL ECG AND TEXT EMBEDDING MODEL

(71) Applicant: AliveCor, Inc., Mountain View, CA (US)

(72) Inventors: Daniel Treiman, San Bruno, CA (US); Joel Q. Xue, Germantown, WI (US)

(21) Appl. No.: 17/585,358

(22) Filed: Jan. 26, 2022

(51) Int. Cl. G16H 50/20; G16H 50/70

(52) U.S. Cl. CPC G16H 50/20; G16H 50/70

(57) ABSTRACT

Embodiments of the present disclosure provide systems and methods for performing an ECG search based on a dual ECG and text embedding model. A text machine learning (ML) model may be trained to generate a text embedding based on a received text representation of an ECG diagnosis. The text ML model may be used to train an ECG encoding ML model to generate an ECG embedding based on received ECG leads data. A database may be populated with a plurality of ECG embeddings, each of the plurality of ECG embeddings generated based on ECG leads data of previously diagnosed ECGs. In response to receiving a query ECG, the ECG ML model may generate a query embedding and a similarity score between the query embedding and each of the plurality of ECG embeddings may be determined. The top K results may be sorted based on similarity score, and may be displayed/visualized.

### TECHNICAL FIELD

Aspects of the present disclosure relate to electrocardiogram (ECG) interpretation, and in particular to search and classification of ECGs to aid in ECG interpretation.

### BACKGROUND

Cardiovascular diseases are the leading cause of death in the world. In 2008, 30% of all global death can be attributed to cardiovascular diseases. It is also estimated that by 2030, over 23 million people will die from cardiovascular diseases annually. Cardiovascular diseases are prevalent across populations of first and third world countries alike, and affect people regardless of socioeconomic status.

Arrhythmia is a cardiac condition in which the electrical activity of the heart is irregular or is faster (tachycardia) or slower (bradycardia) than normal. Although many arrhythmias are not life-threatening, some can cause cardiac arrest and even sudden cardiac death. Indeed, cardiac arrhythmias are one of the most common causes of death when travelling to a hospital. Atrial fibrillation (A-fib) is the most common cardiac arrhythmia. In A-fib, electrical conduction through the ventricles of heart is irregular and disorganized. While A-fib may cause no symptoms, it is often associated with palpitations, shortness of breath, fainting, chest pain or congestive heart failure and also increases the risk of stroke. A-fib is usually diagnosed by taking an electrocardiogram (ECG) of a subject. To treat A-fib, a patient may take medications to slow heart rate or modify the rhythm of the heart. Patients may also take anticoagulants to prevent stroke or may even undergo surgical intervention including cardiac ablation to treat A-fib. In another example, an ECG may provide decision support for Acute Coronary Syndromes (ACS) by interpreting various rhythm and morphology conditions, including Myocardial Infarction (MI) and Ischemia.

Often, a patient with A-fib (or other type of arrhythmia) is monitored for extended periods of time to manage the disease. For example, a patient may be provided with a Holter monitor or other ambulatory electrocardiography device to continuously monitor the electrical activity of the cardiovascular system for e.g., at least 24 hours. Such monitoring can be critical in detecting conditions such as acute coronary syndrome (ACS), among others.

The American Heart Association and the European Society of Cardiology recommends that a 12-lead ECG should be acquired as early as possible for patients with possible ACS when symptoms present. Prehospital ECG has been found to significantly reduce time-to-treatment and shows better survival rates. The time-to-first-ECG is so vital that it is a quality and performance metric monitored by several regulatory bodies. According to the national health statistics for 2015, over 7 million people visited the emergency department (ED) in the United States (U.S.) with the primary complaint of chest pain or related symptoms of ACS. In the US, ED visits are increasing at a rate of or 3.2% annually and outside the U.S. ED visits are increasing at 3% to 7%, annually. In ACS ECG interpretation, the most accurate and specific method is to compare a current ECG with a previously recorded ECG of the same patient to see if there are any significant changes in the ST-T segments and the QRS complex.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1A is a block diagram that illustrates an example system, in accordance with some embodiments of the present disclosure.

FIG. 1B illustrates a single dipole heart model with a 12 lead set represented on a hexaxial system, in accordance with some embodiments of the present disclosure.

FIG. 2 is a block diagram that illustrates a cloud services system, in accordance with some embodiments of the present disclosure.

FIG. 3 is a block diagram that illustrates training of a text encoder, in accordance with some embodiments of the present disclosure.

FIG. 4 is a block diagram that illustrates training of an ECG encoder, in accordance with some embodiments of the present disclosure.

FIG. 5 is a block diagram that illustrates a joint embedding space, in accordance with some embodiments of the present disclosure.

FIG. 6A is a block diagram that illustrates performance of an ECG search, in accordance with some embodiments of the present disclosure.

FIG. 6B is a diagram that illustrates a timeline visualization of ECG search results, in accordance with some embodiments of the present disclosure.

FIG. 6C is a diagram that illustrates a timeline visualization of ECG search results, in accordance with some embodiments of the present disclosure.

FIG. 7 is a block diagram that illustrates training a classifier, in accordance with some embodiments of the present disclosure.

FIG. 8A is a flow diagram of a method of performing an ECG search, in accordance with some embodiments of the present disclosure.

FIG. 8B is a flow diagram of a method of performing an ECG classification, in accordance with some embodiments of the present disclosure.

FIG. 9 is a block diagram of an example computing device that may perform one or more of the operations described herein, in accordance with some embodiments of the present disclosure.

### DETAILED DESCRIPTION

Computer-generated ECG interpretations have been used for many years, and many of the systems that generate them operate based on input from experts and predefined sets of criteria. Recently, the use of deep learning models to generate ECG interpretations has been explored but has not been widely applied to actual medical devices and systems. One of the main reasons is that machine learning models such as DNN models are generally a “black box,” in that they provide the interpretation of an ECG, but do not indicate why a certain result was reached. For a comprehensive multi-lead ECG interpretation, there are many classes such as e.g., rhythm and morphology interpretations, which usually require some explanation or reasoning for the final interpretations. This is unlike the simple types of detection performed by smart watches and other wearable devices for e.g., AFIB and sinus rhythm detection. Utilizing machine learning model based ECG interpretation while adding transparent reasoning for interpretation results is a very important task for further expanding the use of machine learning ECG interpretation models to a variety of clinical applications.

The present disclosure addresses the above-noted and other deficiencies by providing systems and methods for performing an ECG search based on a dual ECG and text embedding model. A processing device may train a text machine learning (ML) model to generate a text embedding based on a received text representation of an ECG diagnosis. The processing device may train, using the text ML model, an ECG encoding ML model to generate an ECG embedding based on received ECG leads data, wherein ECG embeddings generated from similar ECG leads data are proximate to each other in vector space. The processing device may populate a database with a plurality of ECG embeddings, each of the plurality of ECG embeddings generated based on ECG leads data of a previously diagnosed ECG. In response to receiving a query ECG, the processing device may generate, using the ECG ML model, a query embedding and may determine a similarity score between the query embedding and each of the plurality of ECG embeddings. The processing device may sort the ECG embeddings in descending order based on similarity score, and may display/visualize (or transmit to the local computing device 120 for display/visualization) the top K results.

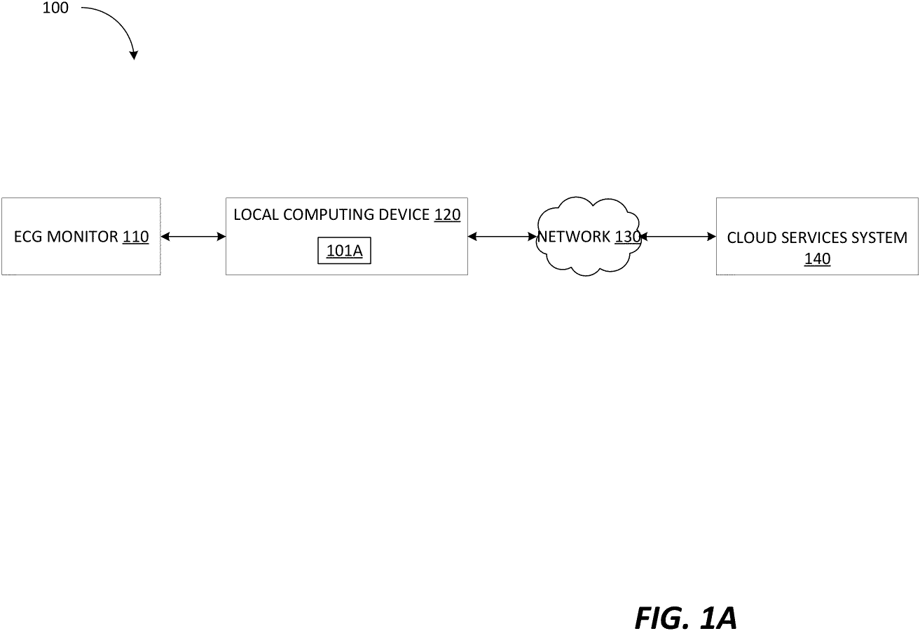

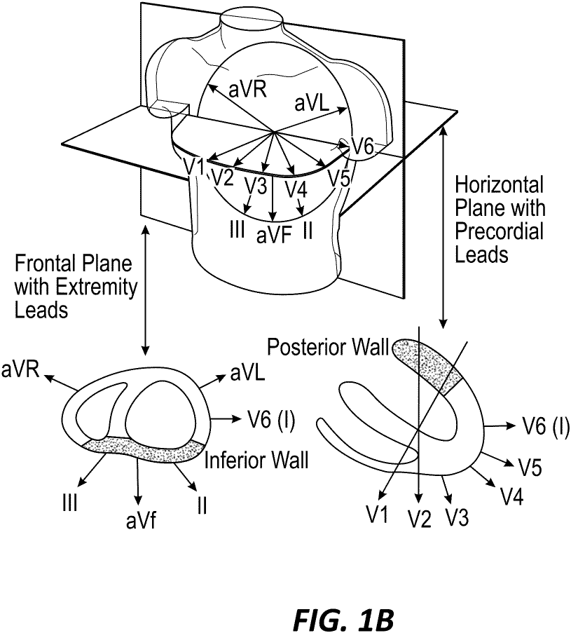

FIG. 1A shows a system 100 in which embodiments of the present disclosure may be realized. The system 100 may be prescribed for use by a first user e.g., by the first user’s physician. Alternatively, system 100 may be used without input from a physician or other third party. The system 100 may comprise a local computing device 120 of the first user. The local computing device 120 may be loaded with a user interface, dashboard, or other sub-system of the cardiac disease management system 100. For example, the local computing device 120 may be loaded with a mobile software application (shown as 101A in FIG. 1) for interfacing with the system 100. The mobile software application 101A may be configured to interface with one or more biometric sensors (e.g., ECG monitor 110) and may comprise software and a user interface for managing biometric data collected by the local computing device 120 from one or more biometric sensors. The local computing device 120 may comprise any appropriate computing device, such as a tablet computer, a smartphone, a server computer, a desktop computer, a laptop computer, or a body-worn computing device (e.g., a smart watch or other wearable), for example. In some embodiments, the local computing device 120 may comprise a single computing device or may include multiple interconnected computing devices (e.g., multiple servers configured in a cluster).

The local computing device 120 may be coupled to one or more biometric sensors. For example, the local computing device 120 may be coupled to an ECG monitor 110 which may comprise a set of electrodes for recording ECG (electrocardiogram) data (also referred to herein as “taking an ECG”) of the first user’s heart. The ECG data can be recorded or taken using the set of electrodes which are placed on the skin of the first user in multiple locations. The electrical signals recorded between electrode pairs may be referred to as leads and FIG. 1B illustrates a 12 lead set comprising the I, II, III, aVR, aVL, aVF, V1, V2, V3, V4, V5, and V6 leads, all represented on a hexaxial system. Varying numbers of leads can be used to record the ECG data, and different numbers and combinations of electrodes can be used to form the various leads. Example numbers of leads used for taking ECGs are 1, 2, 6, and 12 leads. For example, the ECG monitor 110 may be a device comprising 10 electrodes (with six on the user’s chest and one on each of the user’s arms and legs) which may provide a 12-lead ECG. The electrode placed on the right arm may be referred to as RA. The electrode placed on the left arm may be referred to as LA. The RA and LA electrodes may be placed at the same location on the left and right arms, e.g., near the wrist. The leg electrodes may be referred to as RL for the right leg and LL for the left leg. The RL and LL electrodes may be placed on the same location for the left and right legs, e.g., near the ankle.

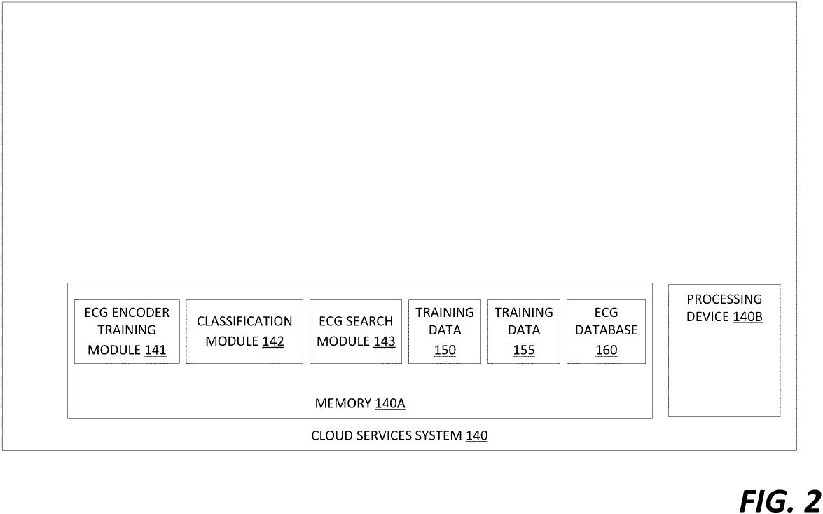

In some embodiments, the ECG monitor 110 may comprise a handheld ECG monitor (such as the KardiaMobile® or KardiaMobile® 6L device from AliveCor® Inc., for example) comprising a smaller number of electrodes (e.g., 2 or 3 electrodes). In these embodiments, the electrodes can be used to measure a subset of the leads illustrated in FIG. 2, such as lead I (e.g., the voltage between the left arm and right arm) contemporaneously with lead II (e.g., the voltage between the left leg and right arm), and lead I contemporaneously with lead V2 or another one of the chest leads such as V5. It should be noted that any other combination of leads is possible. If desired, additional leads can then be algorithmically derived (e.g., by the ECG monitor 110 itself or the local computing device 120) from the determined subset of leads. For example, augmented limb leads can also be determined from the values measured by the LA, RA, LL, and RL electrodes. The augmented vector right (aVR) may be equal to RA - (LA+LL) / 2 or - (I + II) / 2. The augmented vector left (aVL) may be equal to LA - (RA+LL) / 2 or I - II / 2. The augmented vector foot (aVF) may be equal to LL - (RA+LA) / 2 or II - I / 2. In some embodiments, the ECG monitor 110 itself or the local computing device 120 may utilize a machine learning (ML) model to derive the full 12 lead set from a measured subset of leads. In some embodiments, the ECG monitor 110 may be in the form of a smartphone, or a wearable device such as a smart watch. In some embodiments, the ECG monitor 110 may be a handheld sensor coupled to the local computing device 120 with an intermediate protective case/adapter.

The ECG data recorded by the ECG monitor 110 may comprise the electrical activity of the first user’s heart, for example. A typical heartbeat may include several variations of electrical potential, which may be classified into waves and complexes, including a P wave, a QRS complex, a T wave, and sometimes U wave as known in the art. The shape and duration of the P wave can be related to the size of the user’s atrium (e.g., indicating atrial enlargement) and can be a first source of heartbeat characteristics unique to a user.

The duration, amplitude, and morphology of each of the Q, R and S waves can vary in different individuals, and in particular can vary significantly for users having cardiac diseases or cardiac irregularities. For example, a Q wave that is greater than ⅓ of the height of the R wave, or greater than 40 ms in duration can be indicative of a myocardial infarction and provide a unique characteristic of the user’s heart. Similarly, other healthy ratios of Q and R waves can be used to distinguish different users’ heartbeats.

The ECG monitor 110 may be used by the first user to measure their ECG data and transmit the measured ECG data to the local computing device 120 using any appropriate wired or wireless connection (e.g., a Wi-Fi connection, a Bluetooth® connection, a near-field communication (NFC) connection, an ultrasound signal transmission connection, etc.).

The ECG data may be continually recorded by the user at regular intervals. For example, the interval may be once a day, once a week, once a month, or some other predetermined interval. The ECG data may be recorded at the same or different times of days, under similar or different circumstances, as described herein. The ECG data may also be recorded at the same or different times of the interval (e.g., the ECG data may be captured asynchronously). Alternatively, or additionally, the ECG data can be recorded on demand by the user at various discrete times, such as when the user feels chest pains or experiences other unusual or abnormal feelings, or in response to an instruction to do so from e.g., the user’s physician. In another embodiment, ECG data may be continuously recorded over a period of time (e.g., by a Holter monitor or by some other wearable device).

Each ECG data recording may be time stamped and may be annotated with additional data by the user or health care provider to describe user characteristics. For example, the local computing device 120 (e.g., the mobile app 101A thereof) may include a user interface for data entry that allows the user to enter their user characteristics including e.g., a user ID. The local computing device 120 may append the user characteristics to the ECG data and transmit the ECG data to the cloud services system 140.

The ECG data can be transmitted by the local computing device 120 to the cloud services system 140 for storage and analysis. The transmission can be real-time, at regular intervals such as hourly, daily, weekly and/or any interval in between, or can be on demand. The local computing device 120 and the cloud services system 140 may be coupled to each other (e.g., may be operatively coupled, communicatively coupled, may communicate data/messages with each other) via network 130. Network 130 may be a public network (e.g., the internet), a private network (e.g., a local area network (LAN) or wide area network (WAN)), or a combination thereof. In one embodiment, network 130 may include a wired or a wireless infrastructure, which may be provided by one or more wireless communications systems, such as a Wi-Fi hotspot connected with the network 130 and/or a wireless carrier system that can be implemented using various data processing equipment, communication towers (e.g., cell towers), etc. The network 130 may carry communications (e.g., data, message, packets, frames, etc.) between the local computing device 120 and the cloud services system 140.

Machine learning (ML) models are well suited for continuous monitoring of one or multiple criteria to identify anomalies or trends, big and small, in input data as compared to training examples used to train the model. The ML models described herein may be trained on ECG data from a population of users, and/or trained on other training examples to suit the design needs for the model. Machine learning models that may be used with embodiments described herein include by way of example and not limitation: Bayes, Markov, Gausian processes, clustering algorithms, generative models, kernel and neural network algorithms. Some embodiments utilize a machine learning model based on a trained neural network (e.g., a trained recurrent neural network (RNN) or a trained convolution neural network (CNN)).

For example, an ML model may comprise a trained CNN ML model that takes input data (e.g., ECG data) into convolutional layers (aka hidden layers), and applies a series of trained weights or filters to the input data in each of the convolutional layers. The output of the first convolutional layer is an activation map, which is the input to the second convolution layer, to which a trained weight or filter (not shown) is applied, where the output of the subsequent convolutional layers results in activation maps that represent more and more complex features of the input data to the first layer. After each convolutional layer a non-linear layer (not shown) is applied to introduce non-linearity into the problem, which nonlinear layers may include an activation function such as tanh, sigmoid or ReLU. In some cases, a pooling layer (not shown) may be applied after the nonlinear layers, also referred to as a downsampling layer, which basically takes a filter and stride of the same length and applies it to the input, and outputs the maximum number in every sub-region the filter convolves around. Other options for pooling are average pooling and L2-normalization pooling. The pooling layer reduces the spatial dimension of the input volume reducing computational costs and to control overfitting. The final layer(s) of the network is a fully connected layer, which takes the output of the last convolutional layer and outputs an n-dimensional output vector representing the quantity to be predicted. This may result in a predictive output. The trained weights may be different for each of the convolutional layers.

To achieve real-world prediction/detection, a neural network needs to be trained on known data inputs or training examples, thereby resulting in a trained CNN. To train a CNN, many different training examples (e.g., ECG data from users) are input into the model. A skilled artisan in neural networks will fully understand the description above provides a somewhat simplistic view of neural networks to provide some context for the present discussion and will fully appreciate the application of any neural network alone or in combination with other neural networks or other entirely different machine learning models will be equally applicable and within the scope of some embodiments described herein.

FIG. 2 illustrates the cloud services system 140 in accordance with some embodiments of the present disclosure. As shown in FIG. 2, the cloud services system 140 may be a computing device that includes hardware such as processing device 140B (e.g., processors, central processing units (CPUs)), memory 140A (e.g., random access memory (RAM)), storage devices (e.g., hard-disk drive (HDD), solid-state drive (SSD), etc.), and other hardware devices (e.g., sound card, video card, etc.). In some embodiments, memory 140A may be a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. Memory 140A may be configured for long-term storage of data and may retain data between power on/off cycles of the cloud services system 140. The memory 140A may store the ECG data accumulated over time for the user as well as a multitude of other users. The ECG data accumulated over time for a particular user may form a time series health record for that particular user. The cloud services system 140 may comprise any suitable type of computing device or machine that has a programmable processor including, for example, a server computer, a desktop computer, laptop computer, tablet computer, smartphone, etc. In some embodiments, the cloud services system 140 may comprise a single computing device or may include multiple interconnected computing devices (e.g., multiple servers configured in a cloud storage cluster).

The memory 140A may further include an ECG encoder training module 141 and an ECG search module 143, each of which may be executed by the processing device 140B in order to perform some of the functions described herein. The processing device 140B may execute the ECG encoder training module 141 in order to train an ECG encoder for use with the ECG search module 143 as described in further detail herein. The memory 140A may further include training data 150 which may comprise text representations of a plurality of ECG diagnoses for use in training a text encoder 145 as discussed in further detail herein. The memory 140A may further include training data 155 which may comprise ECG recordings (i.e., raw leads data) and text representations of a corresponding diagnosis for each of a plurality of ECGs. As used herein, an ECG recording may refer to the raw leads data of an ECG.

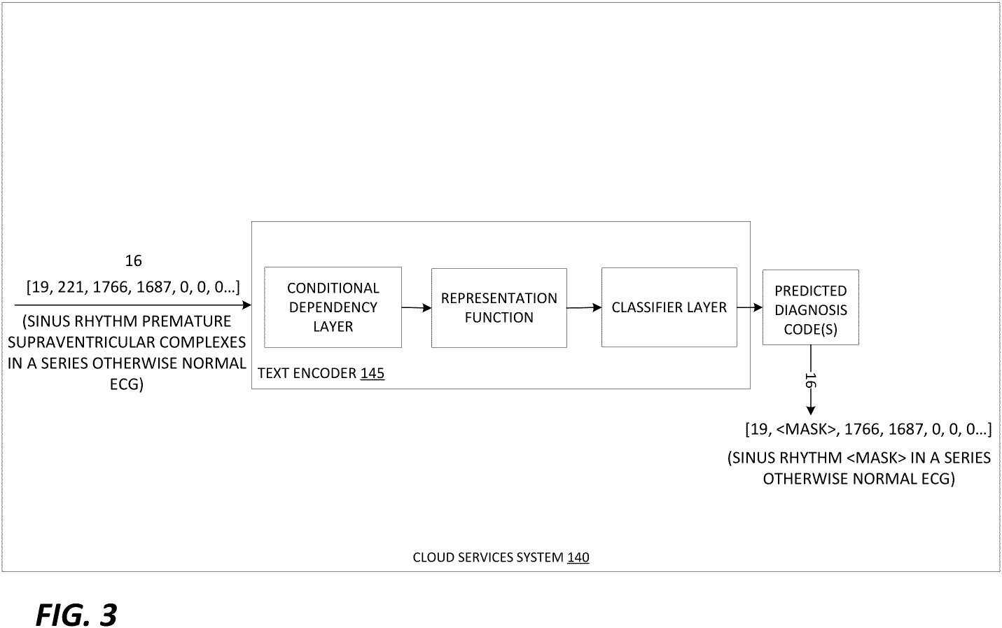

Upon executing the ECG encoder training module 141, the processing device 140B may train a text encoder 145 (shown in FIG. 3) in a semi-supervised manner based on training data 150 to learn a representation function that transforms a text representation of an ECG diagnosis into a vector in an embedding space (referred to herein as a text embedding). The training data 150 may comprise a text representation of each of a plurality of ECG diagnoses as discussed in further detail herein. The text encoder 145 may be any appropriate ML model that can extract learnable, transferrable representations from sequences such as e.g., a bidirectional encoder representations from transformers (BERT) sequence-to-sequence model. An embedding space is a relatively low-dimensional space comprising a learned continuous vector representation of discrete variables into which high-dimensional vectors may be translated. Semi-supervised (or self-supervised learning) involves training an ML model using a limited set of labels, or no labels at all to deduce patterns from the data on which the ML model is trained. In some cases, semi-supervised learning may involve utilization of a proxy task, as discussed in further detail herein.

Embeddings make it easier to perform machine learning on large inputs such as sparse vectors representing words and can be learned and reused across models. The text representation of each ECG diagnosis of the training data 150 may be an ordered sequence of diagnosis codes representing the diagnosis generated for the ECG. Each diagnosis code in a sequence may represent observed conditions (e.g., code 22 = normal sinus rhythm), grammatical modifiers (ex. code 179 = and), and adverbs/adverbial phrases (ex. code 211 = with occasional). For example, the diagnosis code sequence [19, 221, 1766] translates to “sinus rhythm with premature ventricular complexes.” Although the embodiments of the present disclosure are described using an ordered sequence of diagnosis codes representing an ECG diagnosis as the text representation of the ECG diagnosis for example purposes, they are not limited in this way and may be realized using any appropriate text representation of ECG diagnoses.

FIG. 3 illustrates the process of training the text encoder 145. The text encoder 145 may receive as an input, a first sequence of diagnosis codes corresponding to an ECG diagnosis from the training data 150. The first sequence of diagnosis codes may be [19, 221, 1766, 1687, 0, 0, 0...] representing a diagnosis of “sinus rhythm premature supraventricular complexes in a series otherwise normal ECG.” The processing device 140B may remove extraneous information from the sequence of diagnosis codes such as free text portions and date, etc. The number of diagnosis codes/sequence length in the input sequence and the output sequence has been set to 16 as shown in FIG. 3, and may be arbitrarily chosen. As can be seen, the first sequence of diagnosis codes may be padded with zeros (be [19, 221, 1766, 1687, 0, 0, 0...]) to meet the 16 code length. In some embodiments, the number of diagnosis codes in the input and output sequences may be selected to obtain ideal performance.

The text encoder 145 may learn to encode sequences of diagnosis codes into a text embedding (vector in an embedding space) by training on a masked prediction task. Thus, for the first sequence of diagnosis codes, the processing device 140B may remove a diagnosis code from the sequence at random, and replace it with a <MASK> token (i.e., “mask” that diagnosis code) as shown in FIG. 3. In the example of FIG. 3, the diagnosis code representing “premature supraventricular complexes” is masked and the text encoder 145 is trained to predict the masked diagnosis code (e.g., via a classifier layer of the text encoder 145) by outputting a sequence of probability distributions over possible diagnosis codes that can fit in the masked token. Because most masked prediction examples have multiple possible completions, it is reasonable to expect that given enough sequences of diagnosis codes from the training data 150, the text encoder 145 (via e.g., a conditional dependency layer) will learn a representation function that captures the conditional dependencies between the different diagnosis codes and encodes the similarity of related ECG diagnoses. Continuing the example of FIG. 3, the text encoder 145 may come to understand that although more than one diagnosis code can potentially appear after “sinus rhythm,” the phrase “in a series” indicates a high probability of a potential VTAC, and thus the mask would most likely be filled by “premature ventricular complexes.” The text encoder 145 may assign other phrases such as “sinus complexes” and other possibilities a lower probability.

It should be noted that what the text encoder 145 is really learning is a probability distribution of different diagnosis codes that could fit in the masked token which ultimately informs how sequences of diagnosis codes are to be understood/interpreted. More specifically, the representation function of the text encoder 145 may map a sequence of diagnosis codes to a text embedding (vector), and a classifier layer of the text encoder 145 may map a text embedding to a probability distribution of tokens. The classifier layer may be trained to predict the masked diagnosis code from the representation function’s embedding at the position of the masked diagnosis code. Because the diagnosis code in that position is masked, the representation function must generate this embedding from context (i.e., by using unmasked diagnosis codes in the sequence). Contexts which produce similar distributions are likely to have similar embeddings. For example, assume that there are two training instances: “normal sinus rhythm, normal ECG” and “sinus rhythm, normal ECG,” and that the second diagnosis code of each is masked (“normal sinus rhythm, <MASK>” and “sinus rhythm, <MASK>”). The text encoder 145 is likely to learn that “normal sinus rhythm” and “sinus rhythm” are similar (and produce similar context embeddings), since “normal ECG” is the target prediction for both.

Upon completion of the training of the text encoder 145, the text encoder 145 may receive a sequence of diagnosis codes and output a sequence of vectors (continuous real numbers) that capture all the diagnostic info that a physician or health care professional requires, and does so in such a way that similar diagnoses are close together in embedding space.

An ECG search may be implemented by training an ECG encoder 147 to learn a representation function that transforms an (e.g., 10 second 12-lead) ECG recording into a vector in an embedding space (referred to as “ECG embedding”). The ECG encoder 147 should have the same property as the text encoder 145 in that ECG’s with similar diagnoses will be pushed into same region of embedding space, and ECGs with different diagnoses will be pushed away into different regions. Thus, the processing device 140B may train the ECG encoder 147 using a joint embedding space between ECG recordings and text representations of corresponding diagnoses. To do this, the processing device 140B may use the representation function learned by the text encoder 145 to supervise the training of the ECG encoder 147. However, the processing device 140B may utilize a soft form of supervision that merely uses text embeddings as a starting point to learn joint embedding. The processing device 140B may train the ECG encoder 147 using training data 155 which may comprise ECG recordings (i.e., raw leads data) and text representations (i.e., sequences of diagnosis codes) of a corresponding diagnosis for each of a plurality of ECGs.

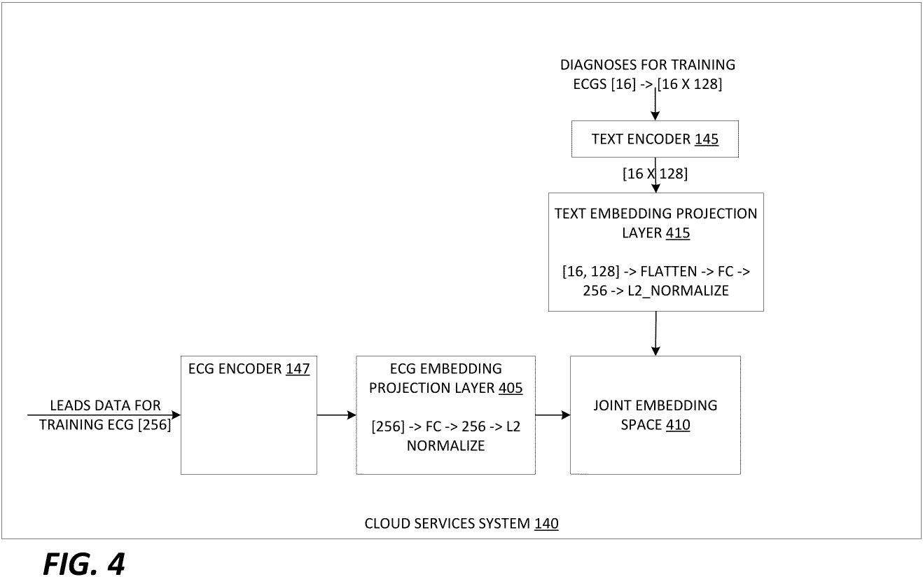

FIG. 4 illustrates the training of the ECG encoder 147. The ECG encoder 147 may receive (from the training data 155) an ECG recording comprising raw ECG leads data, and may utilize multiple layers, where each layer down samples the raw ECG leads data and adds more info/channels A channel is a dimension that represents a feature of the input at some point in time. Typical convolutional networks transform the input by down sampling in the time dimension, and optionally increasing the number of channels. For example, the input (raw ECG leads data) may have dimensions of 3000 (time steps) x 1 (channel), a first layer of the network may output the raw ECG leads data having dimensions of 1500 x 32, a second layer of the network may output the raw ECG leads data having dimensions of 750 x 64, and a third layer of the network may output the raw ECG leads data having dimensions of 375 x 128. As we progress deeper in the network, the raw ECG leads data will have more channels and fewer time steps. The network may transform local information into global information. Finally, the time dimension may be reduced to 1, and each output may represent global information about the raw ECG leads data.

The ECG encoder 147 may comprise a convolutional network, a lead combiner, and a convolutional residual network (not shown in the FIGS.). The convolutional network may down sample and extract features from each lead independently (performing the same operation on each lead). The lead combiner may integrate and mix information from all of the leads. The convolutional residual network may perform additional processing and down sampling, using a technique sometimes referred to as an information bottleneck, wherein information is passed through a smaller space, thereby forcing the ECG encoder 147 to learn how to represent that information more efficiently and discard information that is extraneous or irrelevant. In this way, the processing device 140B may train the ECG encoder 147 to learn how to represent the raw leads data of each ECG of the training data 155 more efficiently and discard information that is unnecessary (as ECG recordings often have a significant amount of redundant information). In some embodiments, during training the processing device 140B may randomly zero out individual leads with 10% probability so as to make the ECG encoder 147 more robust to the effects of a bad lead contact and/or missing or corrupted lead data. Dropping out entire leads encourages the ECG encoder 147 to learn lead-independent features, rather than correlating its output strongly to one “best” lead or a subset of the “best” leads. The processing device 140B may train an ECG embedding projection layer 405 which may comprise a learnable linear transformation which transforms the output sequence of the ECG encoder 147 (ECG embedding) to the joint embedding space 410. The ECG embedding projection layer 405 may comprise a fully-connected layer (not shown) that outputs a vector of 256 length (the size of the joint embedding space 410). The ECG embedding projection layer 405 may also divide the output vector by its Euclidean normal (i.e., L2 normalize the output vector) so that the output vector is always a vector of unit length.

As shown in FIG. 4, the shape of the output of the text encoder 145 is a [16 x 128] array representing 16 embedding vectors each of length 128. The processing device 140B may train a text embedding projection layer 415 to transform the output sequence (text embedding) of the text encoder 145 to the joint embedding space 410. The text embedding projection layer 415 may comprise a fully connected layer (not shown) requiring a one dimensional input and thus the text embedding projection layer 415 may flatten the output of the text encoder 145 to make it one dimensional. The fully connected layer may transform the output sequence (text embedding) of the text encoder 145 to the joint embedding space 410 by outputting a vector of 256 length (the size of the joint embedding space 410). The text embedding projection layer 405 may also L2 normalize the output vector so that the output vector is always a vector of unit length. For every ECG of the training data 155, the text encoder 145 may take the corresponding sequence of diagnosis codes as an input and generate text embeddings (as discussed hereinabove), while the ECG encoder 147 takes the corresponding raw leads data as input, and generates ECG embeddings (as discussed hereinabove). The processing device 140B may project the text embeddings and ECG embeddings (both L2-normalized to unit length) into the joint embedding space 410 using their respective fully-connected layers (ECG embedding projection layer 405 and text embedding projection layer 415).

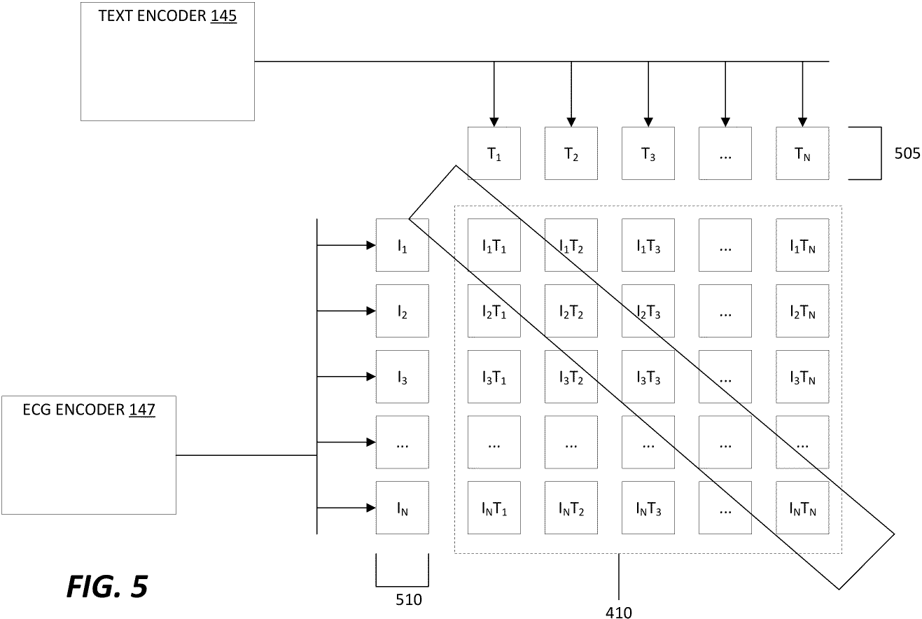

The joint embedding space 410 is where the processing device 140B (executing ECG encoder training module 141) may apply a loss function for training the ECG encoder 147 so that it can learn to match ECG embeddings with corresponding text embeddings. FIG. 5 illustrates a matrix representing the joint embedding space 410. For each training ECG, the text encoder 145 may encode the sequence of diagnosis codes representing the diagnosis of the training ECG into a text embedding, while the ECG encoder 147 may encode the raw leads data of the training ECG into an ECG embedding. As shown in FIG. 5, the text embedding of each training ECG is represented by T₁ - T_{N} across the row 505 while the ECG embedding of each training ECG is represented by I₁ - I_{N} in the column 510. The processing device 140B may utilize the loss function to train the ECG encoder 147 to maximize the similarity between matching ECG and text embeddings, and minimize the similarity between different ECG and text embeddings, thereby training the ECG encoder 147 and the text embedding projection layer simultaneously. More specifically, for each training ECG, the processing device 140B may take the dot product of the corresponding ECG embedding and text embedding. The training objective is to make all diagonal entries (corresponding to the dot product between matching text and ECG embeddings) as close to 1 as possible. Upon training the ECG encoder 147 as discussed hereinabove, the ECG encoder 147 may be able to map pairs of similar ECGs to embeddings with high similarity and map pairs of dissimilar ECGs to embeddings with low similarity.

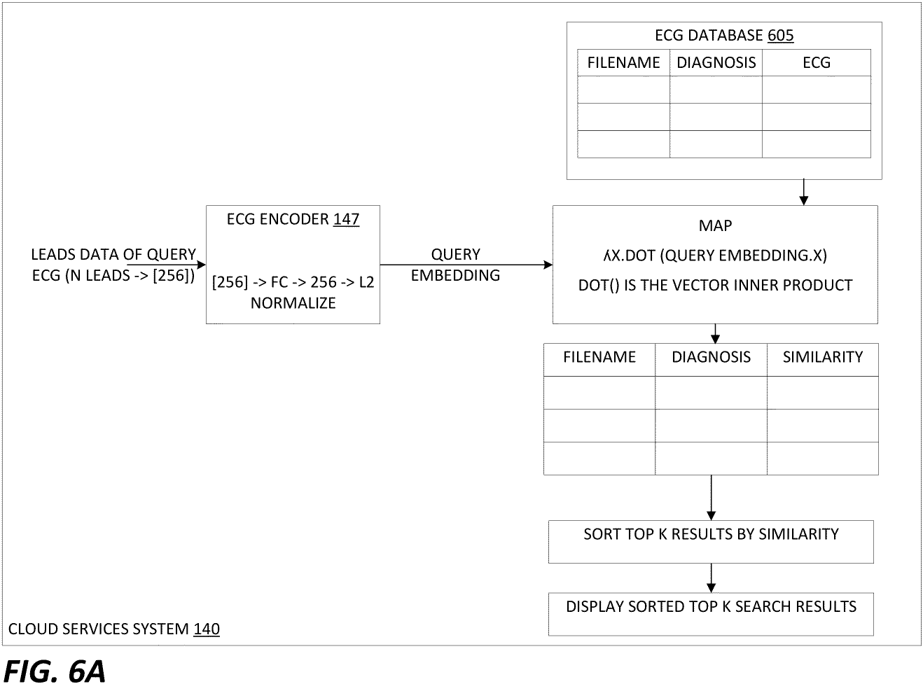

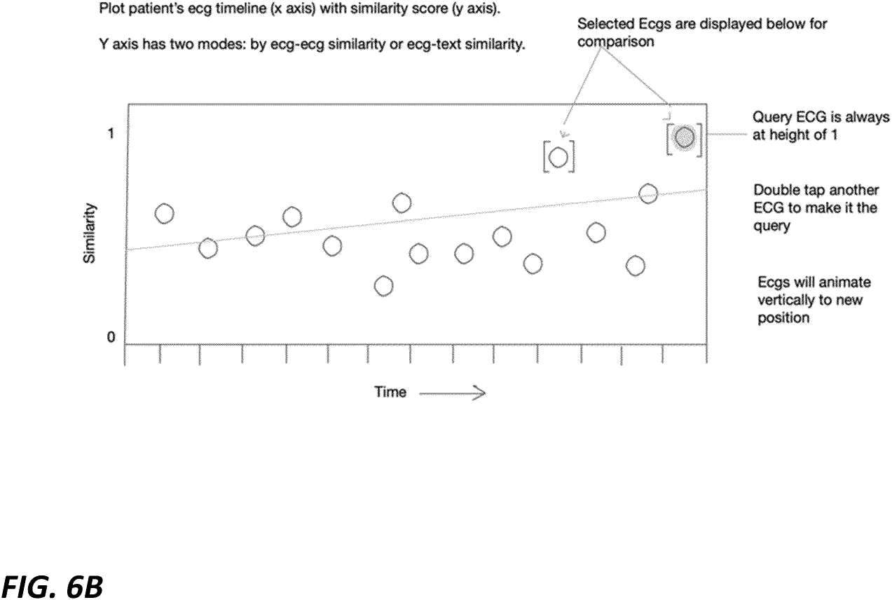

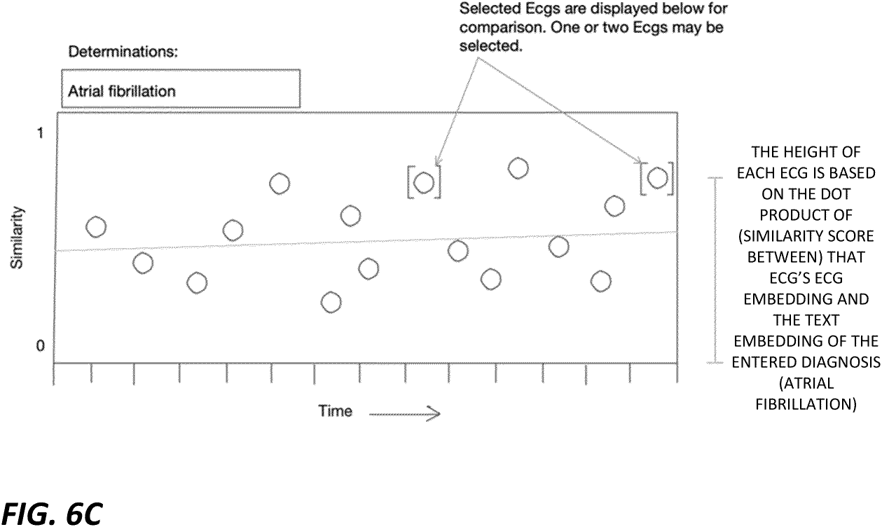

Upon receiving a query ECG from the user (e.g., via local computing device 120 as discussed herein), the processing device 140B may execute the ECG search module 143 in order to utilize the trained ECG encoder 147 to perform an ECG search. FIG. 6 illustrates the process of performing an ECG search. The processing device 140B may prepare a searchable database 605 of ECG embeddings for each ECG in the ECG database 160 (which comprises a plurality of previously recorded ECGs and text representations of their diagnoses) by using the ECG encoder 147 to create ECG embeddings for each ECG in the ECG database 160. It should be noted that the ECG database 160 may comprise previous ECGs of a particular patient, for analysis purposes as discussed herein with respect to FIGS. 6B and 6C. As shown in FIG. 6A, the database 605 may include the filename, diagnosis, and ECG embedding for each of the ECGs in the ECG database 160. To search the database 605 for ECGs similar to the query ECG, the query ECG is encoded by the ECG encoder 147 to create a query embedding.

The processing device 140B (executing ECG search module 143) may compute a similarity score between the query embedding and each ECG embedding in the database 605. ECG embedding vectors have 256 components, are normalized to unit length, and pairs of vectors may be compared using the vector dot product as a metric. Thus, the processing device 140B may use the dot product between the query embedding and an ECG embedding as the similarity score and may compute a similarity score for the query embedding and each ECG embedding. In some embodiments, the similarity scores can be computed quickly and in parallel using a distributed query engine such as Presto or Spark. The processing device 140B may sort the records in descending order based on similarity score, and may display/visualize (or transmit to the local computing device 120 for display/visualization) the top K results.

The use of a dual embedding model to perform an enhanced ECG search may be used in a variety of ways. In one example, the embodiments of the present disclosure may be used to find ECGs that are similar to a selected ECG (e.g., to determine whether a particular patient has had an ECG like the selected one before). In another example, the embodiments of the present disclosure may be used to identify trends, changes, and/or seasonality in a particular user’s cardiac health (e.g., to determine if an ECG is normal for a particular patient, or if there has been a change in their ECG that requires further analysis). In line with these examples, in some embodiments the processing device 140B may generate and display a timeline view of a patient’s ECG history, which may allow the user (e.g., a physician or nurse) to rapidly identify ECGs of interest. The user can select one ECG or a pair which will be displayed below the timeline, either as a single ECG or two ECGs side-by-side for comparison. FIGS. 6B and 6C illustrate different “modes” of time line views where each circle icon may represent an ECG recording, the X axis may represent time, and the Y axis may represent the ECG embeddings (including those based on ECGs in the searchable database 605 and the query embedding based on the query ECG) projected to one dimension. The two modes illustrated in FIGS. 6B and 6C differ in how the Y axis is defined as discussed in further detail herein.

The timeline view of a patient’s ECG records can be used for serial comparison, where the first step is to determine if a significant change has occurred in the rhythm and/or morphology of the patient’s ECG records. A threshold of significant change can be established from the correlation of the dual-embedding variables. If the correlation is higher than the threshold, there is no significant change between the current ECG and the referenced one, so the interpretation status will not change. If the correlation is lower than the threshold, this may indicate that some significant changes have occurred. A further analysis on ECG parameters and embedding variables can define what type of changes have occurred, like ST-T change for an ACS case, or QRS duration change for a bundle branch block case, etc.

FIG. 6B illustrates the timeline view in an “ECG-ECG” mode, where the Y axis may represent the similarity score between each ECG in the searchable database 605 and the query (reference) ECG (i.e., the similarity score between the respective embeddings thereof). The most recent (i.e., query) ECG may be the default reference ECG, but a user can designate a new reference ECG by interacting with the appropriate icon for the ECG they wish to designate as the reference ECG (e.g., double-clicking). All other ECGs in the timeline (other than the reference ECG) may be positioned vertically according to their similarity to the reference ECG.

FIG. 6C illustrates the timeline view in an “ECG-TEXT” mode, where the Y axis may represent the similarity score between a text representation of a diagnosis and each ECG in the searchable database 605 (i.e., the similarity score between the respective text/ECG embeddings thereof). The user may choose or type a diagnosis into the provided data entry area 630 (“atrial fibrulation” as shown in the example of FIG. 6C), and each ECG in the ECG database 160 may be projected onto the Y axis based on their similarity to the provided diagnosis. The text embedding for ‘normal sinus rhythm’ may map to 0 on the Y axis by default. This allows the user to find potentially abnormal ECGs quickly, and also see trends at a glance. As can be seen in FIG. 6C, the height of each ECG in the searchable database 605 on the Y axis may correspond to the dot product of (similarity score between) that ECG’s ECG embedding and the text embedding of the entered diagnosis.

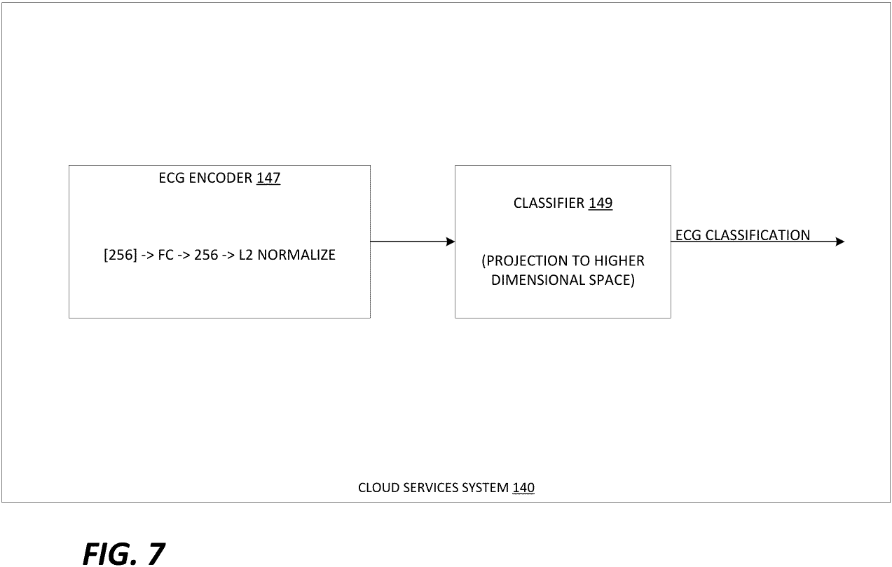

There may be situations where it is desirable to be able to focus on a specific number of conditions when performing an ECG search. For example, a data scientist may wish to mine a database of ECGs for records that may have a diagnosis. In another example, a physician may wish to search a patient’s ECG history, which is particularly relevant in the context of mobile/at home ECG users who often have many unlabeled ECGs. However, because the ECG encoder 147 is trained based on matching text embeddings as discussed above and without reference to any specific classification goal, execution of the ECG search module 143 may result in results that are more generic (and not focused on particular conditions). Thus, in some embodiments, the processing device 140B may execute classification module 142 (instead of ECG search module 143) in order to further train a classifier 149 to classify ECG search results that meet specific conditions that a user is trying to classify for as shown in FIG. 7. FIG. 7 illustrates the process of training the classifier 149 to classify ECG search results focused on specific conditions that the user is trying to classify for. The processing device 140B (executing classification module 142) may use the output of the ECG encoder 147 as input to the classifier 149 in order to train it. The classifier 149 may be a simple ML model. For example, in some embodiments the classifier 149 may simply perform linear regression based on input data from the ECG encoder 147. The classifier 149 may be ideal in situations where it can be trained on a smaller sample set of high quality data (i.e., data that is well labeled). Indeed, such embodiments may allow the processing device 140B to use the pre-trained ECG encoder 147 as a backbone for transfer learning. More specifically, the ECG encoder may pre-process a small sample of ECG data to generate ECG embeddings. A small scale classifier can then be trained on (embedding, label) pairs. This will require less training data and use fewer parameters than directly training on (ECG, label) pairs.

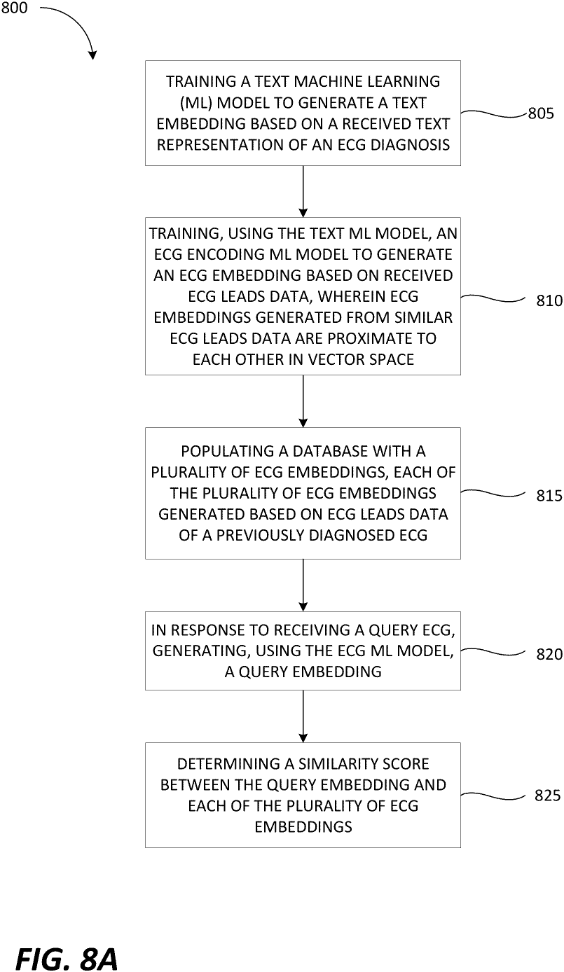

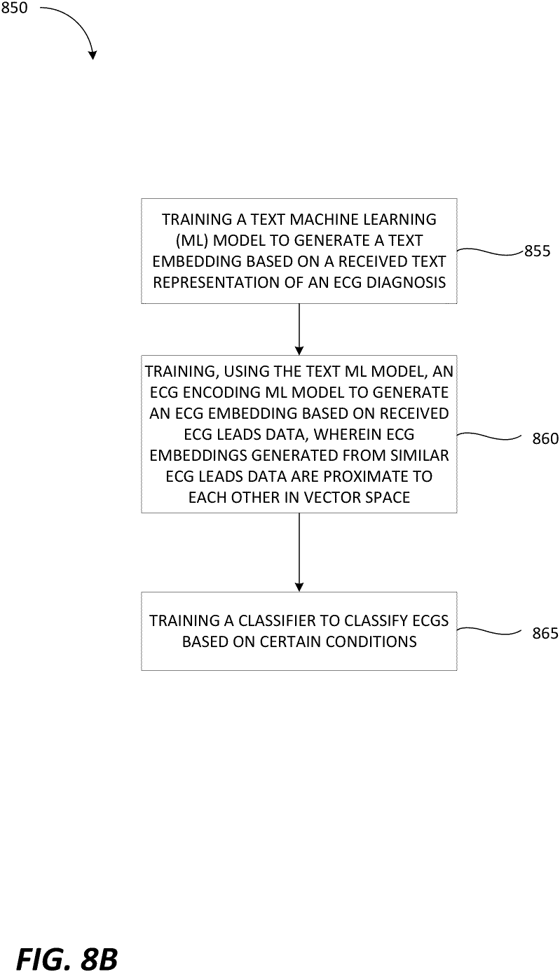

FIG. 8A is a flow diagram of a method 800 for performing an ECG search, in accordance with some embodiments of the present disclosure. Method 800 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the method 800 may be performed by a computing device (e.g., cloud services system 140 illustrated in FIG. 2).

Referring simultaneously to FIGS. 2 and 3 as well, the method 800 begins at block 805, where upon executing the ECG encoder training module 141, the processing device 140B may train a text encoder 145 (shown in FIG. 3) in a semi-supervised manner based on training data 150 to learn a representation function that transforms a text representation of an ECG diagnosis into a vector in an embedding space (referred to herein as a text embedding). The training data 150 may comprise a text representation of each of a plurality of ECG diagnoses as discussed in further detail herein. The text encoder 145 may be any appropriate ML model that can extract learnable, transferrable representations from sequences such as e.g., a bidirectional encoder representations from transformers (BERT) sequence-to-sequence model.

FIG. 3 illustrates the process of training the text encoder 145. The text encoder 145 may receive as an input, a first sequence of diagnosis codes corresponding to an ECG diagnosis from the training data 150. The first sequence of diagnosis codes may be [19, 221, 1766, 1687, 0, 0, 0...] representing a diagnosis of “sinus rhythm premature supraventricular complexes in a series otherwise normal ECG.” The processing device 140B may remove extraneous information from the sequence of diagnosis codes such as free text portions and date, etc. The number of diagnosis codes/sequence length in the input sequence and the output sequence has been set to 16 as shown in FIG. 3, and may be arbitrarily chosen. As can be seen, the first sequence of diagnosis codes may be padded with zeros (be [19, 221, 1766, 1687, 0, 0, 0...]) to meet the 16 code length. In some embodiments, the number of diagnosis codes in the input and output sequences may be selected to obtain ideal performance.

The text encoder 145 may learn to encode sequences of diagnosis codes into a vector in an embedding space (a text embedding) by training on a masked prediction task. Thus, for the first sequence of diagnosis codes, the processing device 140B may remove a diagnosis code from the sequence at random, and replace it with a <MASK> token as shown in FIG. 3. In the example of FIG. 3, the diagnosis code representing “premature supraventricular complexes” is masked and the text encoder 145 is trained to predict the masked diagnosis code (e.g., via a classifier layer of the text encoder 145) by outputting a sequence of probability distributions over possible diagnosis codes that can fit in the masked space. Because most masked prediction examples have multiple possible completions, it is reasonable to expect that given enough sequences of diagnosis codes from the training data 150, the text encoder 145 (via e.g., a conditional dependency layer) will learn a representation function that captures the conditional dependencies between the different diagnosis codes and encodes the similarity of related ECG diagnoses. Continuing the example of FIG. 3, the text encoder 145 may come to understand that although more than one diagnosis code can potentially appear after “sinus rhythm,” the phrase “in a series” indicates a high probability of a potential VTAC, and thus the mask would most likely be filled by “premature ventricular complexes.” Other phrases that could fit into the <MASK> token would be assigned a lower probability. It should be noted that what the text encoder 145 is really learning is a probability distribution of different codes that could fit in the masked slot which ultimately informs how sequences of diagnosis codes are to be understood/interpreted.

Upon completion of the training of the text encoder 145, the text encoder 145 may receive a sequence of diagnosis codes and output a sequence of vectors (continuous real numbers) that capture all the diagnostic info that a physician or health care professional requires, and does so in such a way that similar diagnoses are close together in embedding space.

At block 810, the processing device 140B may train an ECG encoder 147 to learn a representation function that transforms an (e.g., 10 second 12-lead) ECG recording into a vector in an embedding space (referred to as “ECG embedding”). The ECG encoder 147 should have the same property as the text encoder 145 in that ECG’s with similar diagnoses will be pushed into same region of embedding space, and ECGs with different diagnoses will be pushed away into different regions. Thus, the processing device 140B may train the ECG encoder 147 using a joint embedding space between ECG recordings and text representations of corresponding diagnoses. To do this, the processing device 140B may use the representation function learned by the text encoder 145 to supervise the training of the ECG encoder 147. However, the processing device 140B may utilize a soft form of supervision that merely uses text embeddings as a starting point to learn joint embedding. The processing device 140B may train the ECG encoder 147 using training data 155 which may comprise ECG recordings (i.e., raw leads data) and text representations (i.e., sequences of diagnosis codes) of a corresponding diagnosis for each of a plurality of ECGs.

FIG. 4 illustrates the training of the ECG encoder 147. The ECG encoder 147 may receive (from the training data 155) an ECG recording comprising raw ECG leads data, and may utilize multiple layers, where each layer down samples the raw ECG leads data and adds more info/channels. This concept may be referred to as an information bottleneck, wherein information is passed through a smaller space, thereby forcing the ECG encoder 147 to learn how to represent that information more efficiently and discard information that is extraneous or irrelevant. In this way, the processing device 140B may train the ECG encoder 147 to learn how to represent the raw leads data of each ECG of the training data 155 more efficiently and discard information that is unnecessary (as ECG recordings often have a significant amount of redundant information). For every ECG of the training data 155, the text encoder 145 may take the corresponding sequence of diagnosis codes as an input and generate text embeddings, while the ECG encoder 147 takes the corresponding raw leads data as input, and generates ECG embeddings. The processing device 140B may project the text embeddings and ECG embeddings (both L2-normalized to unit length) into the joint embedding space 410 using fully-connected layers.

The joint embedding space 410 is where the processing device 140B (executing ECG encoder training module 141) may apply a loss function for training the ECG encoder 147 so that it can learn to match ECG embeddings with corresponding text embeddings. FIG. 5 illustrates a matrix representing the joint embedding space 410. For each training ECG, the text encoder 145 may encode the sequence of diagnosis codes representing the diagnosis of the training ECG into a text embedding, while the ECG encoder 147 may encode the raw leads data of the training ECG into an ECG embedding. As shown in FIG. 5, the text embedding of each training ECG is represented by T₁ - T_{N} across the row 505 while the ECG embedding of each training ECG is represented by I₁ - I_{N} in the column 510. The processing device 140B may utilize the loss function to train the ECG encoder 147 to maximize the similarity between matching ECG and text embeddings, and minimize the similarity between different ECG and text embeddings, thereby training the ECG encoder 147 and the text embedding projection layer simultaneously. More specifically, for each training ECG, the processing device 140B may take the dot product of the corresponding ECG embedding and text embedding. The training objective is to make all diagonal entries (corresponding to the dot product between matching text and ECG embeddings) as close to 1 as possible. Upon training the ECG encoder 147 as discussed hereinabove, the ECG encoder 147 may be able to map pairs of similar ECGs to embeddings with high similarity and map pairs of dissimilar ECGs to embeddings with low similarity.

At block 815, the processing device 140B may prepare a searchable database 605 of ECG embeddings for each ECG in the ECG database 160 (which comprises a plurality of previously recorded ECGs and text representations of their diagnoses) by using the ECG encoder 147 to create ECG embeddings for each ECG in the ECG database 160. As shown in FIG. 6, the database 605 may include the filename, diagnosis, and ECG embedding for each of the ECGs in the ECG database 160. At block 820, upon receiving a query ECG, the query ECG is encoded by the ECG encoder 147 to create a query embedding. At block 825, the processing device 140B (executing ECG search module 143) may compute a similarity score between the query embedding and each ECG embedding in the database 605. ECG embedding vectors have 256 components, are normalized to unit length, and pairs of vectors may be compared using the vector dot product as a metric. Thus, the processing device 140B may use the dot product between the query embedding and an ECG embedding as the similarity score and may compute a similarity score for the query embedding and each ECG embedding. In some embodiments, the similarity scores can be computed quickly and in parallel using a distributed query engine such as Presto or Spark. The processing device 140B may sort the ECG embeddings in descending order based on similarity score, and may display/visualize (or return to the local computing device 120 for display/visualization) the top K results.

FIG. 8B is a flow diagram of a method 850 of performing ECG classification, in accordance with some embodiments of the present disclosure. Method 850 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the method 850 may be performed by a computing device (e.g., cloud services system 140 illustrated in FIG. 3).

There may be situations where it is desirable to be able to focus on a specific number of conditions when performing an ECG search. However, because the ECG encoder 147 is trained based on matching text embeddings as discussed above and without reference to any specific classification goal, execution of the ECG search module 143 may result in results that are more generic (and not focused on particular conditions). The method 850 may begin at block 855 and 860, which are similar to blocks 805 and 810 described above with respect to FIG. 8A. At block 865, the processing device 140B may execute classification module 142 (instead of ECG search module 143) in order to further train a classifier 149 to classify ECG search results that meet specific conditions that a user is trying to classify for as shown in FIG. 7. FIG. 7 illustrates the process of training the classifier 149 to classify ECG search results focused on specific conditions that the user is trying to classify for. The processing device 140B (executing classification module 142) may use the output of the ECG encoder 147 as input to the classifier 149 in order to train it. The classifier 149 may be a simple ML model. For example, in some embodiments the classifier 149 may simply perform linear regression based on input data from the ECG encoder 147. The classifier 149 may be ideal in situations where it can be trained on a smaller sample set of high quality data (i.e., data that is well labeled).

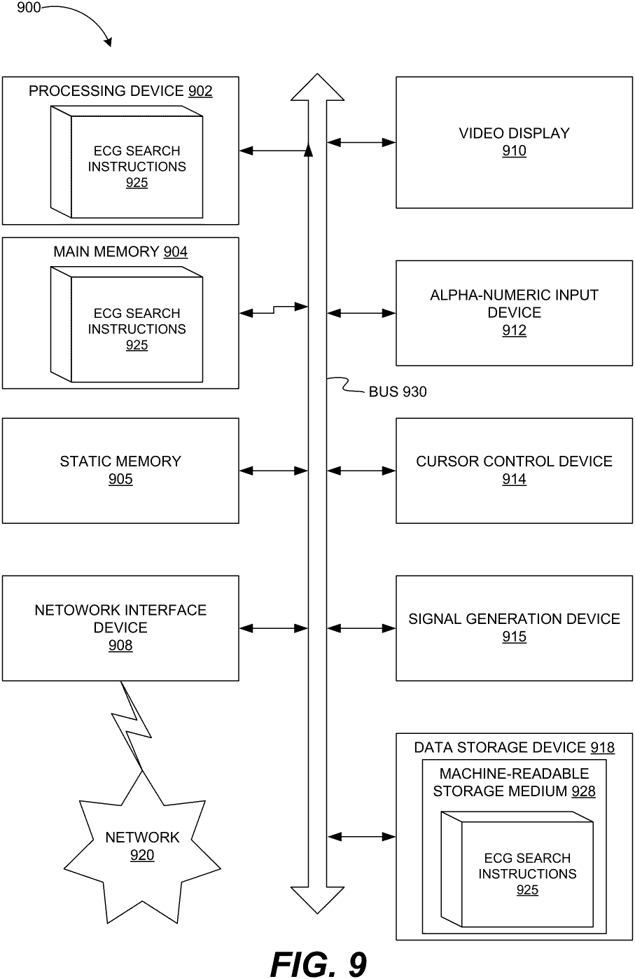

FIG. 9 illustrates a diagrammatic representation of a machine in the example form of a computer system 900 within which a set of instructions, for causing the machine to perform any one or more of the methodologies discussed herein for performing an ECG search.

In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 900 may be representative of a server.

The exemplary computer system 900 includes a processing device 902, a main memory 904 (e.g., read-only memory (ROM), flash memory, dynamic random access memory (DRAM), a static memory 906 (e.g., flash memory, static random access memory (SRAM), etc.), and a data storage device 918, which communicate with each other via a bus 930. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Computing device 900 may further include a network interface device 908 which may communicate with a network 920. The computing device 900 also may include a video display unit 910 (e.g., a liquid crystal display (LCD) or a cathode ray tube (CRT)), an alphanumeric input device 912 (e.g., a keyboard), a cursor control device 914 (e.g., a mouse) and an acoustic signal generation device 916 (e.g., a speaker). In one embodiment, video display unit 910, alphanumeric input device 912, and cursor control device 914 may be combined into a single component or device (e.g., an LCD touch screen).

Processing device 902 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 902 may also be one or more special-purpose processing devices such as an application specific integrated circuit (ASIC), a field programmable gate array (FPGA), a digital signal processor (DSP), network processor, or the like. The processing device 902 is configured to execute ECG search instructions 925, for performing the operations and steps discussed herein.

The data storage device 915 may include a machine-readable storage medium 928, on which is stored one or more sets of ECG search instructions 925 (e.g., software) embodying any one or more of the methodologies of functions described herein. The ECG search instructions 925 may also reside, completely or at least partially, within the main memory 904 or within the processing device 902 during execution thereof by the computer system 900; the main memory 904 and the processing device 902 also constituting machine-readable storage media. The ECG search instructions 925 may further be transmitted or received over a network 920 via the network interface device 908.

While the machine-readable storage medium 928 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof.

What is claimed is:

1. A method comprising:
training a text machine learning (ML) model to generate a text embedding based on a received text representation of an ECG diagnosis;
training, using the text ML model, an ECG encoding ML model to generate an ECG embedding based on received ECG leads data such that ECG embeddings generated from similar ECG leads data are proximate to each other in vector space;
populating a database with a plurality of ECG embeddings, each of the plurality of ECG embeddings generated based on ECG leads data of a previously diagnosed ECG;
in response to receiving a query ECG, generating, using the ECG ML model, a query embedding; and
determining a similarity score between the query embedding and each of the plurality of ECG embeddings.

2. The method of claim 1, further comprising:
ranking each of the plurality of ECG embeddings based on a respective similarity score between the ECG embedding and the query embedding; and
displaying a number of the ECG embeddings having highest similarity scores in descending order.

3. The method of claim 1, wherein training the ECG ML model using the text ML model comprises:
generating, by the text ML model, a training text embedding based on a text representation of a diagnosis of each of a plurality of training ECGs to create a plurality of training text embeddings;
generating, by the ECG ML model, a training ECG embedding based on leads data for each of the plurality of training ECGs to create a plurality of training ECG embeddings;
projecting the plurality of training ECG embeddings and the plurality of training text embeddings into a joint embedding space; and
utilizing a loss function to train the ECG ML model to match each training ECG embedding with a corresponding training text embedding.

4. The method of claim 3, wherein utilizing the loss function to train the ECG ML model comprises:
for each of the plurality of training ECG embeddings, determining a dot product of the training ECG embedding with each of the training text embeddings, wherein the ECG ML model is to make the dot product between a training ECG embedding and a training text embedding of a training ECG as close to one as possible.

5. The method of claim 1, wherein text representations of an ECG diagnosis comprise a set of diagnosis codes.

6. The method of claim 5, wherein training the text ML model comprises:
for a text representation of each of a plurality of training ECG diagnoses:
randomly removing a diagnosis code from a corresponding set of diagnosis codes of the training ECG diagnosis and replacing the removed diagnosis code with a mask token; and
training the text ML model to predict the removed diagnosis code based on remaining diagnosis codes of the set of diagnosis codes.

7. The method of claim 1, wherein the text ML model is a bidirectional encoder representations from transformers (BERT) model.

8. A system comprising:
an electrocardiogram (ECG) monitor to record ECG data of a user; and
a cloud services system to:
train a text machine learning (ML) model to generate a text embedding based on a received text representation of an ECG diagnosis;
train, using the text ML model, an ECG encoding ML model to generate an ECG embedding based on received ECG leads data such that ECG embeddings generated from similar ECG leads data are proximate to each other in vector space;
populate a database with a plurality of ECG embeddings, each of the plurality of ECG embeddings generated based on ECG leads data of a previously diagnosed ECG;
in response to receiving a query ECG, generate, using the ECG ML model, a query embedding; and
determine a similarity score between the query embedding and each of the plurality of ECG embeddings.

9. The system of claim 8, wherein the cloud services system is further to:
rank each of the plurality of ECG embeddings based on a respective similarity score between the ECG embedding and the query embedding; and
display a number of the ECG embeddings having highest similarity scores in descending order.

10. The system of claim 8, wherein to train the ECG ML model using the text ML model, the cloud services system is to:
generate, by the text ML model, a training text embedding based on a text representation of a diagnosis of each of a plurality of training ECGs to create a plurality of training text embeddings;
generate, by the ECG ML model, a training ECG embedding based on leads data for each of the plurality of training ECGs to create a plurality of training ECG embeddings;
project the plurality of training ECG embeddings and the plurality of training text embeddings into a joint embedding space; and
utilize a loss function to train the ECG ML model to match each training ECG embedding with a corresponding training text embedding.

11. The system of claim 10, wherein to utilize the loss function to train the ECG ML model, the cloud services system is to:
for each of the plurality of training ECG embeddings, determine a dot product of the training ECG embedding with each of the training text embeddings, wherein the ECG ML model is to make the dot product between a training ECG embedding and a training text embedding of a training ECG as close to one as possible.

12. The system of claim 8, wherein text representations of an ECG diagnosis comprise a set of diagnosis codes.

13. The system of claim 12, wherein to train the text ML model, the cloud services system is to:
for a text representation of each of a plurality of training ECG diagnoses:
randomly remove a diagnosis code from a corresponding set of diagnosis codes of the training ECG diagnosis and replacing the removed diagnosis code with a mask token; and
train the text ML model to predict the removed diagnosis code based on remaining diagnosis codes of the set of diagnosis codes.

14. The system of claim 8, wherein the text ML model is a bidirectional encoder representations from transformers (BERT) model.

15. A non-transitory computer-readable medium having instructions stored thereon which, when executed by a processing device, cause the processing device to:
train a text machine learning (ML) model to generate a text embedding based on a received text representation of an ECG diagnosis;
train, using the text ML model, an ECG encoding ML model to generate an ECG embedding based on received ECG leads data such that ECG embeddings generated from similar ECG leads data are proximate to each other in vector space;
populate a database with a plurality of ECG embeddings, each of the plurality of ECG embeddings generated based on ECG leads data of a previously diagnosed ECG;
in response to receiving a query ECG, generate, using the ECG ML model, a query embedding; and
determine a similarity score between the query embedding and each of the plurality of ECG embeddings.

16. The non-transitory computer-readable medium of claim 15, wherein the processing device is further to:
rank each of the plurality of ECG embeddings based on a respective similarity score between the ECG embedding and the query embedding; and
display a number of the ECG embeddings having highest similarity scores in descending order.

17. The non-transitory computer-readable medium of claim 15, wherein to train the ECG ML model using the text ML model, the processing device is to:
generate, by the text ML model, a training text embedding based on a text representation of a diagnosis of each of a plurality of training ECGs to create a plurality of training text embeddings;
generate, by the ECG ML model, a training ECG embedding based on leads data for each of the plurality of training ECGs to create a plurality of training ECG embeddings;
project the plurality of training ECG embeddings and the plurality of training text embeddings into a joint embedding space; and
utilize a loss function to train the ECG ML model to match each training ECG embedding with a corresponding training text embedding.

18. The non-transitory computer-readable medium of claim 17, wherein to utilize the loss function to train the ECG ML model, the processing device is to:
for each of the plurality of training ECG embeddings, determine a dot product of the training ECG embedding with each of the training text embeddings, wherein the ECG ML model is to make the dot product between a training ECG embedding and a training text embedding of a training ECG as close to one as possible.

19. The non-transitory computer-readable medium of claim 15, wherein text representations of an ECG diagnosis comprise a set of diagnosis codes.

20. The non-transitory computer-readable medium of claim 19, wherein to train the text ML model, the processing device is to:
for a text representation of each of a plurality of training ECG diagnoses:
randomly remove a diagnosis code from a corresponding set of diagnosis codes of the training ECG diagnosis and replacing the removed diagnosis code with a mask token; and
train the text ML model to predict the removed diagnosis code based on remaining diagnosis codes of the set of diagnosis codes.
